(19) United States
(12) Patent Application Publication
(10) Pub. No.: US 2024/0335459 A1
(43) Pub. Date: Oct. 10, 2024

(54) THERAPEUTIC USES AND METHODS OF DELIVERY OF VISCOUS COMPOSITIONS DURING PRE AND POST SURGICAL PERIODS, FOR TREATMENT OF RESPONSIVE DISEASES OF THE EAR, NOSE AND THROAT

(71) Applicant: Topical Sinus Therapeutics, Inc., Camarillo, CA (US)

(72) Inventors: Russell N. REITZ, Camarillo, CA (US); John C. Tarrant, Camarillo, CA (US)

(21) Appl. No.: 18/400,993

(22) Filed: Dec. 29, 2023

Related U.S. Application Data
(63) Continuation of application No. 17/024,576, filed on Sep. 17, 2020, now abandoned.
(60) Provisional application No. 62/901,744, filed on Sep. 17, 2019.

(51) Int. Cl. A61K 31/58; A61B 17/24; A61K 9/00; A61K 9/06; A61K 9/50; A61K 31/7036; A61K 47/36; A61K 47/38; A61M 1/00; A61M 3/02; A61M 25/10; A61M 31/00

(52) U.S. Cl. CPC A61K 31/58; A61B 17/24; A61K 9/0043; A61K 9/06; A61K 9/5015; A61K 31/7036; A61K 47/36; A61K 47/38; A61M 3/0262; A61M 31/00; A61B 2017/246; A61M 1/84; A61M 25/10; A61M 2210/0618; A61M 2210/0681

(57) ABSTRACT

A medication or combination of medications for the treatment of a disease involving the sinuses is incorporated into a sterile medical gel. The viscosity of the gel or the adhesion of the gel to the patient's sinus cavity allows the gel to be maintained in the cavity for a sufficient length of time for treatment of the affected area, thereby reducing the amount of wasted medication that would otherwise result from other topical methods. In one embodiment, the medicated gel comprises a sterile viscous gel having a viscosity sufficient to be maintained within a paranasal sinus cavity after insertion, and a medication dispersed in the sterile viscous gel.

### RELATED APPLICATIONS

This application is a continuation of U.S. application Ser. No. 17/024,576 filed on Sep. 17, 2020 which claims the benefit of U.S. Provisional Application No. 62/901,744 filed on Sep. 17, 2019, the contents of which are incorporated herein by reference in their entirety.

### TECHNICAL FIELD

The present disclosure relates generally to drug administration, and more particularly, some embodiments relate to viscous topical compositions for drug administration.

### BACKGROUND

Sinusitis is an inflammation of the paranasal sinuses. One of the problems associated with current treatments for chronic sinusitis is the delivery of medications in sufficient concentrations into the sinuses and having it remain in contact with the mucosal lining for a sufficient period of time to promote healing. The small sinus structures are hollow cavities with narrow openings where an infection may easily adhere to the lining of the sinuses and are difficult to reach. Oral medications (after absorption) and intravenous medications must pass from the blood stream into the sinus cavity(s) in sufficient concentrations and for a specific duration of time to fully eliminate the sinus infection. Often, using oral and intravenous drug therapy, it is extremely difficult to achieve sufficient drug concentration in the sinuses without causing specific side effects. With oral and intravenous therapy, the drug travels to all areas of the body, this can negatively affect certain tissues, including certain organs and organ systems not intended to be affected.

There are numerous examples where a drug is metabolized in the liver only to be discontinued upon a rise in liver enzymes. In other cases, the kidneys are unable to timely remove the medication from the blood causing an increase in serum concentration over time. Increased serum concentration may lead to tissue and organ toxicities. Also, oral or intravenous administration of medication especially antibiotics, corticosteroids, or antifungals used to treat chronic sinusitis may lead to severe gastrointestinal side effects, resulting in premature discontinuation of the prescribed medication.

Typical treatments of sinus infection include the following methods of delivery (a) intravenous drug administration; (b) oral drug administration; (c) nasal sprays; (d) nebulization; (e) sinus irrigations or rinses; (f) pulsating irrigation; (g) atomization; and (h) drug-eluting devices.

Intravenous drug administration is usually very expensive. Depending on the prescribed drug and duration of therapy, a usual course of intravenous antibiotics/antifungals, etc. may cost between $2,500 and $21,000 dollars. Also, many intravenous drug therapies require management by monitoring serum drug concentrations to prevent tissue/organ damage. Furthermore, elevated serum levels of certain medications can lead to renal and/or ototoxicity.

Oral drug administration often has gastrointestinal side effects, or lack of gastrointestinal absorption. Other organ system side effects may include bone, tendon, or muscle tissue damage with extreme pain. Furthermore, oral drug administration can also result in insomnia, agitation and acute/chronic diarrhea.

Nasal sprays typically have a large particle size that may prevent penetration into the sinuses.

Nebulization often has lengthy treatment times, results in poor concentration of drug into the sinuses, and has possible pulmonary drug deposition.

Sinus irrigations or rinses suffer from poor compliance because they are not comfortable for patients. Moreover, studies indicate up to 97% of the medicated irrigation is wasted rushing out of the sinuses and nasal cavity. Furthermore, residual pockets of fluid may be a cause of more frequent infections. Pulsating irrigation devices suffer from the same drug waste and residual gel pockets as seen with sinus irrigations or rinses.

Atomizers provide a small volume of medication that may not penetrate into sinuses. In addition, short pulse atomization is often not sufficient in duration to penetrate or power into sinuses.

Drug-eluting devices left within the sinuses may impede wound healing. In particular, because these drug-eluting devices remain attached to the sinus wall, they interfere with certain steps of post-operative protocol designed to promote wound healing. For example, debridement of damaged or infected tissue to improve the healing potential of the remaining healthy tissue cannot be adequately performed while the drug-eluting device is in place. Accordingly, the effectiveness of wound healing via drug-eluting devices is significantly reduced despite the localized administration of medication into the sinus. Furthermore, bioabsorbable drug eluding stents are known to elude medication more rapidly than designed and not absorb properly leaving behind fishbone-like particles, which have to be surgically removed at a later time.

### SUMMARY

In accordance with one or more embodiments, various features and functionality can be provided to enable or otherwise facilitate drug delivery using a viscous composition to minimize disadvantages of conventional drug delivery including drug-eluting devices and optimize the therapeutic benefits of the drug.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. The drawings are for the purpose of illustration and description only and are not intended as a definition of the limits of the invention.

### BRIEF DESCRIPTION OF THE DRAWINGS

FIG. 1A illustrates example delivery device, according to an implementation of the disclosure.

FIG. 1B illustrates example cannula for fitting into the delivery device of FIG. 1A, according to an implementation of the disclosure.

FIG. 1C depicts an example cannula in gel communication with a syringe for delivery of the viscous composition to a patient's paranasal sinus cavity, according to an implementation of the disclosure.

FIG. 2 illustrates a medical kit comprising any number of saline syringes, medicated gel syringes for immediate post-operative use, medicated syringes for follow up care visits, and luer-lock cannulas, according to an implementation of the disclosure.

FIG. 3 depicts a bottle with a spray pump or squeeze pump for delivery of the viscous composition to a patient's paranasal sinus cavity, according to an implementation of the disclosure.

FIG. 4 illustrates a vial for delivery by squeezing the contents of the vial directly into the nasal passages, according to an implementation of the disclosure.

FIG. 5 illustrates a swab or pledget that may be dipped into a gel and then spreading using the swab into a patient's paranasal sinus cavity, according to an implementation of the disclosure.

FIG. 6 depicts a tube that may be squeezed for delivery of the viscous composition to a patient's paranasal sinus cavity, according to an implementation of the disclosure.

FIG. 7 depicts a can of compressed gel under pressure for delivery of the viscous composition to a patient's paranasal sinus cavity, according to an implementation of the disclosure.

FIG. 8 depicts a balloon sinuplasty device for delivery of the viscous composition to a patient's paranasal sinus cavity, according to an implementation of the disclosure.

### DETAILED DESCRIPTION

Described herein are viscous compositions for topical drug delivery into paranasal sinuses, methods of using and making them. The details of some example embodiments of the embodiments of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

As alluded to above, paranasal sinus cavities produce the mucus necessary for the nasal passages to work effectively and are prone to infection and inflammation caused by various microorganisms (e.g., bacteria, virus, fungi, etc.) and pollutants and/or allergens. The infection may cause swelling of the epithelial linings of the sinuses and mucus buildup, a condition known as sinusitis. Often, inflammation of the sinus cavities is accompanied by inflammation of the nasal cavities resulting in rhinosinusitis. Certain conditions, such as nasal polyps, or small growths in the nasal passage, or allergies may increase predisposition to inflammation.

Treatment options for sinusitis, rhinosinusitis, and polyps may include administering medications such as antileukotrienes, antibiotics or corticosteroids to help reduce inflammation, treat infection and reduce polyp size. Antihistamines may be administered to reduce allergic reactions. Unfortunately, some patients may not respond to the treatment favorably; resulting in a chronic sinusitis, a condition in which symptoms persist, or continually return, after 12 weeks. Some chronic conditions may require a more invasive treatment, such as surgery directed at removing infected sinus tissue, cartilage or bone to create a wider opening to improve drainage of infected mucus in the sinuses, as well as polyp removal.

The outcome of sinus surgery is often dependent on the immediate postoperative healing phase which requires decreasing the possibility of postoperative infections and re-mucosalization of the sinonasal cavities. Antibiotics are commonly used to reduce microorganism load and minimize infections. As alluded to above, topically administered antibiotics bypass many of the common side effects associated with oral or intravenous administrations and thus are more effective. Notably, traditional methods of administration that include packing materials have been associated with pain and discomfort to the patient as well as additional trauma to the mucosa during removal. While use of a dressing that is absorbable may be appealing, it has been shown to interfere with debridement and saline rinsing procedures necessary for re-mucosalization to occur. In addition, some drug-eluting devices do not fully absorb, resulting in further bleeding, crusting and other complications causing the surgical removal of the partially disintegrated device.

In contrast, a viscous composition (e.g., a hydrogel) is not only effective at administering an antibacterial/anti-inflammatory agent topically for an extended period of time due its adherence properties, it does not interfere with postoperative procedures (e.g., debridement and saline rinsing) and thus aids in improving the surgical outcome. In particular, the viscous medicated composition is a post-operative medicated “salve” that provides a soothing sensation to the patient, temporarily relieving discomfort, which is especially welcome in the post-operative period. Because of the viscosity, the compound can remain in the sinonasal cavity and/or other areas for a longer period thereby prolonging the contact with the injured area, which results in an increased efficacy of the medication giving the medicine. Additionally, the viscous medicated composition may be formulated to include multiple medicated agents designed to provide treatment for a number of symptoms (e.g., inflammation and allergy). The viscous medicated composition is water soluble and has the ability to dissolve or liquify over time and drain away with the body's natural mucus movement, thus eliminating the need to surgically removing the composition. Further, because of its water-soluble properties, the viscous medicated composition may be easily rinsed away, if necessary (e.g., in the event of an adverse reaction). Finally, the viscous medicated composition may be reapplied, as needed either during in-office visit or by the patient themselves.

Accordingly, dressings that comprise a viscous composition for delivering medications to the sinonasal cavities and/or other areas, they provide effective treatment options during the peri- and postoperative periods.

### Viscous Composition

Sinus Gels can be made in various viscosities ranging from a thin lubricant to a thick gel or paste. Viscosities ranges may include centipoise measurements anywhere between 10 to 300,000 centipoise. For example, the viscosity of sinus gels in the 10 cps range would be similar to blood, whereas the viscosity of sinus gels in the 300,000 cps range would be similar to peanut butter (i.e. Sinus gel viscosity in the 70,000 cps to 100,000 cps range would resemble toothpaste).

In some embodiments, a viscous composition configured to deliver a topical medication may comprise a gel preparation having physical characteristics suitable for delivering and maintaining adherence of an active compound at a sinus cell wall. As alluded to above, because the medication is delivered via a gel rather than orally, intravenously, through nebulization, or via a drug-eluting device, in the perioperative period, the surgical outcome is improved.

In some embodiments, a viscous composition may include a hydrogel. Hydrogels may serve as semi-synthetic or synthetic extra cellular matrix that provides an amenable environment for cellular adherence and has the capacity to carry small molecule drugs and/or proteins, growth factors and other necessary components for cell growth. That is, when utilized for drug delivery, hydrogels can localize drugs, increase drugs concentration at the site of action and reduce off-targeted side effects.

In some embodiments, natural hydrogel compositions are often made of polysaccharide or protein chains. For example, polysaccharide-based hydrogels may include hydrogels made of alginate, cellulose (cellulose analogs), chitin, chitosan, dextran, hyaluronic acid, pectin, starch, and xanthan gum. Polysaccharides and glycoproteins may also provide natural moisture and lubricity to the treated area.

In one embodiment, the sinus gel may also include one or more amino acids that promote sinus health such as essential amino acids that the body does not create on its own (i.e. histidine, isoleucine, leucine, lysine, methionine, phenylalanine, threonine, tryptophan, and valine). N-acetylcysteine (NAC) is a modified form of a dietary amino acid that works as an antioxidant in the body. It also helps thin mucus. Other examples may include lysine, arginine oxo-proline, and L-glutamine.

In some embodiments, synthetic hydrogels may include synthetic polymers such as poly (vinyl alcohol), polyacrylamide, poly (ethylene oxide), poly (ethylene glycol) and polylactic acid (polylactide). For example, hydrogels may include methyl cellulose, carboxymethyl cellulose, hydroxyethyl cellulose, hydroxypropyl-methyl cellulose, hydroxypropyl cellulose, and hydroxypropyl methylcellulose (HPMC), known as Hypromellose, or hydroxypropyl methylcellulose (HPMC), as well as carbomer resins 690-941.

In some embodiments, additional compounds may be added to the viscous composition (e.g., a hydrogel). For example, in one embodiment, the viscous composition may comprise a mixture of hydroxyethyl cellulose, water, and propylene glycol. For example, a final volume of a 200 cc may include Hydroxyethyl cellulose in a range of 1 cc to 12 cc, Propylene Glycol in a range of 5 cc to 30 cc, and the remaining volume of distilled or sterile water may range from 100 cc to 175 cc.

In some embodiments, additional compounds added to the hydrogel may affect one or more properties of the viscous composition. For example, some compounds may affect viscosity and adherence properties, longevity in certain environments and interaction with environmental factors such as oxygen, light, heat, and humidity, while other may control the release of the medications carried by the viscous composition such as hydrated silica.

### Chitosan

In some embodiments, chitosan may be included in the viscous composition comprising a hydrogel, as alluded to above. Chitosan is a linear polysaccharide made by treating the chitin shells of crustaceans with an alkaline substance, like sodium hydroxide. Because chitosan is capable of opening tight junctions between epithelial cells of mucosal membranes and improving drug molecules transportation, addition of chitosan makes suitable in paranasal sinus administration. Additionally, chitosan facilitated drug delivery is preferred because of the high rate of water absorption and mucoadhesive potential associated with chitosan.

In some embodiments, estrogen, estrogen analogs, progesterone, and progesterone analogs may be included in the viscous composition comprising a hydrogel. Estrogen and/or progesterone may increase epithelial cell growth of sinus tissues. Additionally, it is known that these two hormones play a significant role in thickening the uterine lining during pregnancy and may play a similar role in other cell lines. Therefore, by including estrogen and/or progesterone in the viscous composition may result in in the thickening of epithelial tissues lining the sinus cavities.

### Compounds Promoting Structure

In some embodiments, additional compounds comprising sealants and/or adhesives may be included in the viscous composition comprising a hydrogel. These ingredients may allow an increased adherence of the hydrophilic gel to the damaged inflamed and infected epithelial tissues of the sinus cavities. Certain adhesives may include a variety of absorbable and nonabsorbable carbomer polymers, such as carbomer homopolymer A. Other tissue adhesants may include certain monomers, such as cyanoacrylate.

In some embodiments, compounds that work as a scaffold and thus support a variety of living tissues may be included in the viscous composition. For example, a fibrin scaffold which is a network of blood proteins such fibrinogen and thrombin that participate in blood clotting may be used. Fibrin, fibrin glue or fibrin sealant may be used to control of gelation times and provide slow-release delivery of medications like antibiotics.

In some embodiments, additional compounds such as protein serum albumin (either bovine or human) may be included in the viscous composition comprising a hydrogel. In some embodiments, the viscous composition may be synthesized from serum albumin.

In some embodiments, additional compounds such as gamma globulin may be included in the viscous composition comprising a hydrogel. For example, immunoglobulins (antibodies) and other gamma globulins which are not immunoglobulins may be included in the viscous composition.

In some embodiments, additional proteins may be included in the viscous composition comprising a hydrogel. For example, engineered or naturally occurring proteins may be included. For example, sericin, a protein created by Bombyx mori (silkworms) in the production of silk, which consists of two proteins, fibroin and sericin may be included. In some embodiments, spider web proteins fibroins or similar proteins of bacterial origin may be included. Furthermore, genetically engineered silk protein combining certain DNA sequences, corticosteroids or antibiotics or antifungals may be used to form a lattice adhering to the damaged tissue.

### Other Compounds

In some embodiments, additional compounds comprising silver (Ag++) or gold (Au++) ions may be included in the viscous composition comprising a hydrogel. In some embodiments, additional compounds comprising salts containing silver or gold ions or colloidal suspensions containing these two ions may be included in the viscous composition comprising a hydrogel. Silver has been demonstrated to have anti-microbial effects and has not been shown to suffer from bacterial resistance. For example, a number of research studies has examined the effectiveness of silver-based products in reducing the bacterial burden and treating wound infection. Similarly, gold ion in solution and or a colloidal suspension is known for its anti-infective and anti-inflammatory properties. Because bacteria present in wounds may have a negative impact on healing, the recent increase in multi-resistant bacterial strains is a cause of concern in wound care. Accordingly, silver may be a useful tool in treating infected wounds and therefore may also be effective in speeding the healing process after sinus surgery.

In some embodiments, additional compounds comprising EDTA may be included in the viscous composition comprising a hydrogel. For example, EDTA (ethylenediaminetetraacetic acid) and its salts, Calcium Disodium EDTA, Diammonium EDTA, Dipotassium EDTA, Disodium EDTA, TEA-EDTA, Tetrasodium EDTA, Tripotassium EDTA and Trisodium EDTA, may be used. Because EDTA catalyzes metal ions, which can be present in any water-based system, addition of EDTA salts may prevent metal ions from reacting with various compounds of the viscous composition and prevent a variety of unfavorable side-effects (e.g., spoilage and rancidity, degradation, haze formation and precipitation, poor foaming and rinsability performance). EDTA has also been shown to have properties that break down biofilm and allow medications to penetrate bacterial colonies more successfully.

In some embodiments, additional compounds comprising Biopolymers compatible with Sinus tissue, such as Carbomer Homopolymer may be included in the viscous composition comprising a hydrogel. Carbomer Homopolymers may provide a layered coating to inflamed tissues. This coating may allow for increased adherence of the medication to the inflamed sinus tissues.

In some embodiments, additional compounds comprising hyaluronic acid (HA) and related compounds may be included in the viscous composition comprising a hydrogel. HA is known to have drug delivery property which facilitate topical drug absorption. Additionally, HA is highly compatible with a variety of biomacromolecules. By including HA in the viscous composition may result in an increased drug penetration into the epithelial tissues.

In some embodiments, viscosity of the composition may be regulated. For example, by adding one or more medications and/or compositions, the viscosity of the composition may be varied. Regulation of viscosity controls the adherence of the composition to the epithelium of the sinus wall. That is, more viscous compositions may have a longer adherence time. Certain viscosity modifiers may include various cellulose polymers such as methylcellulose, carboxymethyl cellulose, hydroxyethyl cellulose, and Hypromellose. Furthermore, other viscosity modifiers may include polyethylene glycol and/or glycerin.

In some embodiments, the viscosity of the composition may be varied based on the medication or other compounds that have been incorporated into the viscous composition.

In some embodiments, the viscosity may be varied based on the requirements of a particular procedure for which compound is used. For example, the viscosity of the compound may vary according to factors such as physician preference, medication administered, patient physiology, desired administration time, administration temperature, individual medications and medication diluents. In some embodiments, typical viscosities of the compound may have a consistency of a petrolatum jelly preparation or a gelatin paste approaching 200,000 centipoise.

Some embodiments may include certain buffers. Such buffers may include phosphate, bicarbonate, citric acid, boric acid and sodium borate. Other embodiments may include various humectants. Such humectants may include glycerin, propylene glycol and xylitol.

Additional embodiments may include a solubility enhancer such as alpha cyclodextrin or beta cyclodextrin. In yet other embodiments, surfactants may be included. Such surfactants may include, for example, polysorbate 20, polysorbate 60 and polysorbate 80. Additionally, some embodiments may include fluids and/or gels such as distilled water, sterile water, isotonic saline, hypotonic saline and hypertonic saline.

### Medications

In one embodiment, a specific medication or combination of medications is incorporated into a medicated gel. In a further embodiment, the gels comprise a specific medication or combination of medications in a water-soluble formula. The viscosity of the gel or the adhesion of the gel to the patient's sinus cavity allows the gel to be maintained in the cavity for a sufficient length of time for treatment of the affected area. This reduces the amount of wasted medication that would otherwise result from other topical methods.

According to another embodiment, the medicated gel is applied via various surgical instruments known in the art directly onto and around the infected sinus anatomy. The route of application is through the nasal cavity. This direct application bypasses the requirement of sufficient serum drug concentration over an extended period of time to treat the chronic sinus infection or related sinuses disease.

In some embodiments of the invention, a sterile gel that solvates a medication is provided for use in inserting the medication into the sinus cavities of a patient.

In some embodiments, a medication is incorporated into the gel. Medications may include medications designed to treat various sinus related diseases, such as sinusitis, rhinitis, biofilm-caused diseases, or sinus polyps. In further embodiments, the gel and medication combination may be used as an aid in surgical procedures, such as surgical suction and surgical debridement. In these procedures, some gels embodiments, especially those containing an anti-inflammatory may be inserted immediately postoperatively to reduce tissue inflammation due to the surgical procedure and to promote tissue healing. Some embodiments, especially those containing an anti-inflammatory may be inserted directly into a sinus cavity containing polyps to reduce the size and possibly with repeated application eliminate the polyp's preventing a surgical procedure.

Medications used in various embodiments may include antimicrobial medications, antibiotic medications, anti-inflammatory medications, anti-fungal medications, nonsteroidal anti-inflammatory drugs (NSAIDS), mucolytic medications, antihistamine medications, decongestant medications, vasoconstrictor medications, anti-viral medications, chelating agent medications, oncologic medications, or combinations thereof. Some particular medications include tobramycin, gentamicin, vancomycin, ceftriaxone, clindamycin, vancomycin, levofloxacin, ciprofloxacin, mupirocin, and corticosteroids. Other medications include drugs within the following antibiotic drug classes: Penicillins (all categories); Cephalosporins (multi-generation); Tetracyclines; Macrolides; Quinolones; Fluoroquinolones; Aminoglycosides; Carbapenems; Sulfonamides, and Polymyxins. In further embodiments, the medications include drugs within the following anti-fungal (azoles) drug classes: Fluconazole, Itraconazole, Voriconazole, Parconazole and Isoconazole. Other embodiments may include Amphotericin B Sulfate, including all lipid, matrix and microsphere forms of Amphotericin such as Ambisome, Abelcet and Amphotec. In still further embodiments, the medications include corticosteroid drugs such as, Mometasone (furoate and furoate monohydrate), betamethasone, fluticasone (furoate and furoate monohydrate), ciclesonide, hydrocortisone and budesonide. In further embodiments, the medications may include leukotriene receptor antagonists such as Montelukast and zafirlukast.

In some embodiments, one or more medications included in the viscous composition may be encapsulated or microencapsulated. For example, the microencapsulation of medications may result in protection of the enclosed product (e.g., due to factors such as insolubility, volatility, reactivity, hygroscopicity, and physical state associated with particular medications, environmental factors such as oxygen, light, heat, and humidity which could destroy any labile compound, or even body's immune response), and controlled release of the encapsulated contents (e.g. by virtue of extended or timed release).

In some embodiments, medications may be encapsulated or microencapsulated using in a lipid matrix, liposomal bead or prepared with a lipid salt attachment. For example, many oncolytic and anti-fungal medications may require encapsulation. In some embodiments, mometasone or any other of medications can be encapsulated or microencapsulated to permit controlled release of the encapsulated contents. The encapsulation may transform the medication into a “time-released” drug which allows for more time for the drug to be in contact with the infected or inflamed area of the sinus tissue and thus provide for a more positive therapeutic outcome.

In some embodiments, one or more naturopathic substances can be included in the viscous substance. In some embodiment, the one or more naturopathic substances may be included along with the medications. In yet other embodiments the one or more naturopathic substances may be delivered via the viscous composition without the medications. For example, substances such as tea tree oil known for its antibacterial and anti-inflammatory properties may be in included in the viscous composition for delivery to the sinuses.

In some embodiments, herbal extracts or essential oils such as cinnamon, pine, spearmint, menthol, eucalyptol, thymol, cajeput, peppermint, wintergreen, benzoin or camphor to may be included provide a natural and soothing feeling to the inflamed tissues of the sinuses. Other ingredients shown to be effective in relieving symptoms of sinusitis include European elder, Rumex acetosa (common sorrel), Primula veris (cowslip), Verbena officinalis (European vervain), Gentiana lutea (gentian). These herbs may work by thinning mucus and helping the sinuses drain. They may also help strengthen the immune system.

In another embodiment, the sinus gel may include Bromelain or Quercetin. Several studies suggest that the enzyme, bromelain may help reduce inflammation and swelling and relieve symptoms of sinusitis. Bromelain is often combined with the flavonoid quercetin, which may act as an antihistamine.

In some embodiments, a gel including xylitol may be applied to the treatment area. Xylitol has been proven to contain antibacterial properties and some studies indicate that xylitol is useful in post-operative ESS and septoplasty and rhinorrhea care.

In some embodiments, active ingredients isolated from the one or more naturopathic substances may be included in the viscous substance. For example, methylglyoxal (MG), an ingredient found in Manuka honey, may be isolated and included in the viscous substance. Manuka honey is known for its antibacterial properties and has been demonstrated to be effective against biofilm-producing bacteria. A number of physicians have prescribed Manuka honey sinus irrigations or rinses for patients with active chronic rhinosinusitis and prior sinus surgery. By including methylglyoxal within the viscous substance allows to deliver the anti-bacterial and anti-inflammatory properties of Manuka honey without the tedious process of performing sinus irrigation. Further, because methylglyoxal exists in in very small quantities in Manuka Honey, isolating it allows to increase the healing effects of Manuka honey.

In some embodiments, the viscous compound may include compounds that provide a timed release of medication. For example, by including Hypromellose related compounds and liposomal soluble microspheres, and/or other lipid formulations and polymer compounds such as polylactic acid and polyglycolic acid. These ingredients allow the viscous compound to provide extended or timed release of the medication carried by the viscous compound.

### Vitamin D3

Further embodiments of the invention may feature compounds or gel formulations that include Vitamin D3. Clinical literature illustrates that Vitamin D3 therapy has certain immunological benefits for patients suffering from chronic sinusitis with nasal polyps [CRSwNP] and patients diagnosed with allergic fungal rhinosinusitis [AFRS]. Chronic sinusitis patients with nasal polyps and allergic fungal sinusitis patients have been found to have insufficient levels of Vitamin D3. In addition, these patients were shown to have increased dendritic cells, which may play a part in the development of sinus tissue inflammation.

Increased Vitamin D3 has been shown in clinical studies to improve a cell's immunological response by lowering circulating dendritic cells in those patients with CRSwNP and AFRS. Immunological researchers have observed these and other benefits as it relates to sinus disease.

Vitamin D therapy has previously been topically administered inside an emollient base carrier for the purpose of alleviating inflammation and epithelial cell irritation. In addition, Vitamin D has been used in combination with other vitamins in various healthcare products such as ointments used in the care of diaper rash patients. One such combination includes Vitamin D and Vitamin A.

In some embodiment, a medicated gel such as described above is modified to include Vitamin D3. In particular, the medical gel includes a micro fine Vitamin D3 suspension that is incorporated inside a hydrophilic carrier gel base. This Vitamin D3 hydrophilic gel may contain concentrations as low as 10 units per cc of Vitamin D3 and concentrations as high as 10,000 units per cc of Vitamin D3.

In some embodiments, the medicated gel may be applied to other areas that require treatment. For example, the external ear canal, the passage between the outer ear and eardrum may become inflamed. The inflammation of the external ear canal, or otitis externa, is often referred to as “swimmer's ear” because repeated exposure to water can make the ear canal more vulnerable to inflammation. The symptoms associated with otitis externa may include ear pain, itchiness in the ear canal, a discharge of liquid or pus from the ear, and some degree of temporary hearing loss. By applying a medicated gel that remains in the ear canal for an extended period of time (with a syringe and canula), inflammation and infection may be reduced more effectively than with current alternative therapies.

### Example Preparation

In an example embodiment, a medical gel comprises a 200-cc preparation of tobramycin/mometasone gel. Compounds used in making the gel include: (a) 2000 mg of Tobramycin (10 mg/cc) (COA=0.676=2,959 mg); (b) 48 mg of mometasone (1200 mcg/5 cc) (COA=1.0=48 mg); (c.) 6 g hydroxyethyl cellulose; (d) 25 cc of 100% ethyl alcohol; (e) 12 cc propylene glycol; (f) sterile water qs for 200 cc; and (g) one drop per 200 cc final volume of polysorbate 80.

In yet another example embodiment, a medicated gel may include diphenhydramine. Diphenhydramine is an antihistamine used to relieve the symptoms of allergy, hay fever, and the common cold caused by body's production histamine or acetylcholine, during an allergic reaction. These symptoms include rash, itching, watery eyes, itchy eyes/nose/throat, cough, runny nose, and sneezing. It is preferable after sinus surgery for the patient not to blow their nose or sneeze, if possible, for a period of time. Using diphenhydramine alone or combined with other medications in a medicated gel may alleviate these symptoms and provide for a better patient outcome. Diphenhydramine works by blocking the substances produced by the body during an allergic reaction. The effects of diphenhydramine include alleviating symptoms by drying watery eyes and runny nose. This medication works by blocking histamine that is created during an allergic reaction.

### Example Protocol

In some embodiments, a protocol may be followed during a postoperative patient care period. For example, the protocol may include administration of sterile saline solution contained in two 20cc sterile saline Luer Lock syringes; use of two catheters of appropriate design; administration of 3 cc of mometasone furoate or mometasone monohydrate contained in two 5 cc syringes; and administration of sterile saline solution contained in one 10 cc sterile luer lock syringe.

In some embodiments, the protocol may include the following steps: (1) Lavage affected sinus cavity(s) with a sterile saline solution contained in a 20 cc sterile saline syringe and cannula; (2) Perform a surgical debridement of the sinus cavity to remove damaged and/or infected tissue; (3) Lavage sinus cavity with a sterile saline solution contained in a 20 cc sterile saline syringe with cannula; (4) Deposit an appropriate amount of mometasone furoate or mometasone monohydrate contained in a viscous substance (e.g., hydrogel) onto the affected sinus tissue with syringe and cannula. (5) Follow up visits may occur in one to five weeks with reapplication of the sinus gel as necessary.

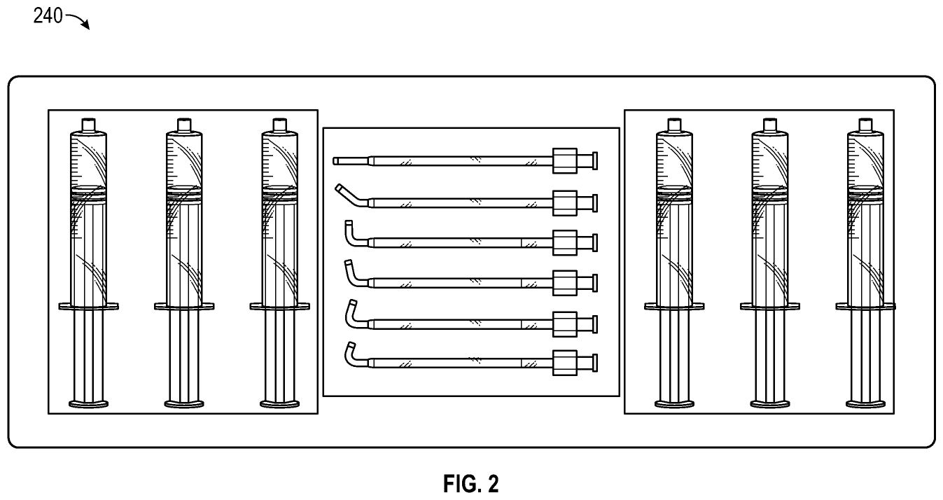

Referring to FIG. 3, in some embodiments patients may apply medicated “Sinus Gel” without the aid of a physician directly to the nasal passages via a bottle 300 with a spray pump or squeeze pump with or without an applicator tip.

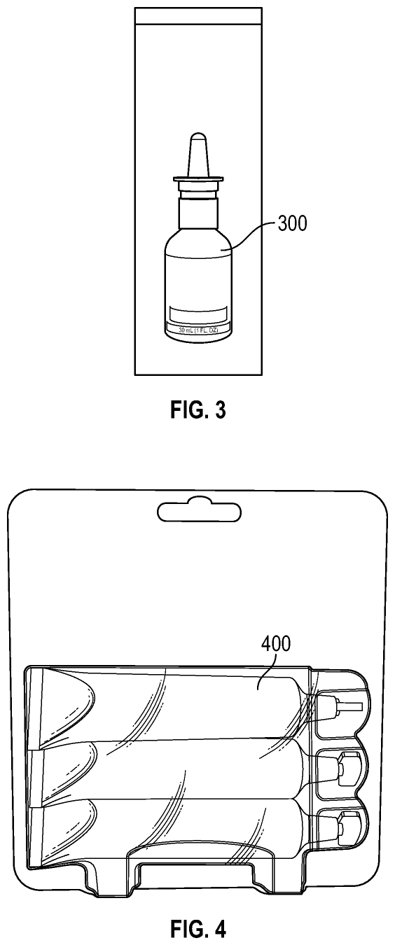

Referring to FIG. 4, in some embodiments patients may apply medicated “Sinus Gel” without the aid of a physician directly to the nasal passages via a vial 400 by squeezing the contents of the vial directly into the nasal passages.

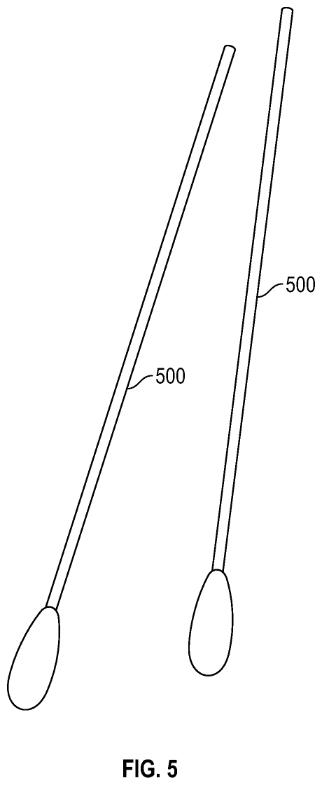

Referring to FIG. 5, in some embodiments patients may apply medicated “Sinus Gel” without the aid of a physician directly to the nasal passages via a swab by dipping a swab or pledget 500 into the gel and then spreading the gel with the swab applicator directly into the nasal passages. One embodiment includes a “gel packet” containing one or two cotton-tip applicators saturated with a sufficient volume of a corticosteroid gel, such as mometasone monohydrate gel. The gel is applied to the nasal polyps reducing inflammation and size. Another example is two pledget type gel swabs 500 per pack, one for each nostril. These would be used by the patient twice per day for 30 days for swabbing the nostrils and/or nasal polyps to reduce inflammation and polyp size. Another example may include a small container (½ oz. to 2 oz.) consisting of a corticosteroid gel, such as mometasone monohydrate gel, using a cotton-tip applicator to apply a sufficient quantity of gel to the nasal polyps reducing inflammation and size. In this scenario the gel and swabs are in separate containers. Or the patient could use their own “Q-tip over the counter” swab. The volume of gel would be sufficient to last at least 30 days in a twice per day (BID) application.

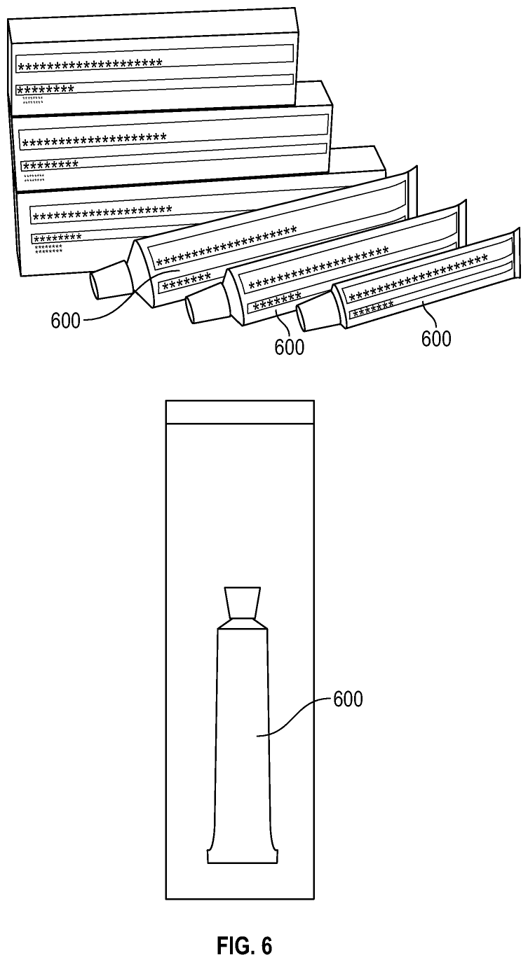

Referring to FIG. 6, in some embodiments patients may apply medicated “Sinus Gel” without the aid of a physician directly to the nasal passages by squeezing a tube 600 with or without an applicator tip attached.

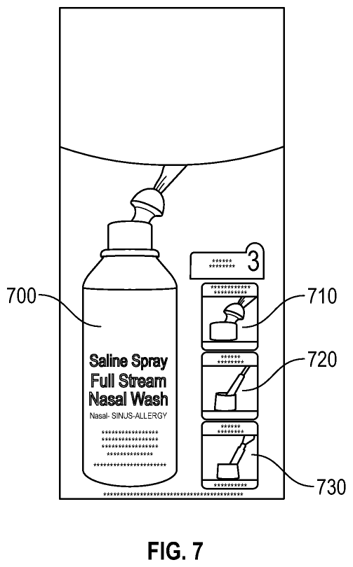

Referring to FIG. 7, in some embodiments patients may apply medicated “Sinus Gel” without the aid of a physician directly to the nasal passages via a can of compressed gel 700 under pressure with or without an applicator tip 710,720,730.

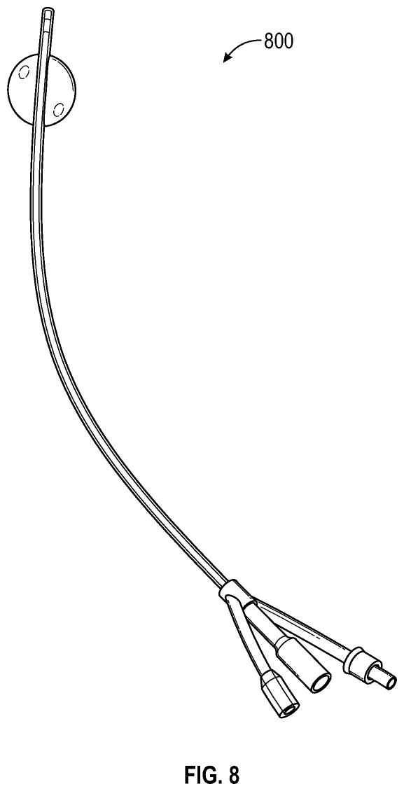

Referring to FIG. 8, in a further surgical implementation, a physician may utilize a balloon sinuplasty device 800 for delivery of the viscous composition to a patient's paranasal sinus cavity. In particular, balloon sinuplasty device 800 includes a squeeze pump or syringe at the proximal end for delivery of the viscous composition to a patient's paranasal sinus cavity from the distal end of device 800. The irrigation catheter is advanced over the sinus guidewire or sinus illumination system into the target sinus.

In some embodiments patients may apply medicated gel without the aid of a physician directly to the nasal passages by inserting a water-soluble formula of the “Sinus Gel” into an irrigation bottle and then diluting the gel with saline, e.g., sterile saline or by mixing a packet or “sachet” of powdered saline and water. After mixing or shaking the solution/suspension, the patient may insert the medicated liquid into the nasal passages by following the directions of the irrigation device of their choice (i.e., a squeeze irrigation bottle, a hydropulse device, Navage® device, a netipot, and/or other similar such device etc.)

### Preparation of Example Gel

Step 1. The measured amount of mometasone is dissolved in 25 cc of ethyl alcohol 100%. The dissolved mometasone is drawn into a 20-cc sterile syringe with a 0.22-micron Millipore disc filter attachment.

Step 2. The measured amount of tobramycin is dissolved in 15 cc of sterile water. The dissolved tobramycin is drawn into a 35-cc sterile syringe with a 0.22-micron Millipore disc filter attachment.

Step 3. 150 cc of sterile water is measured and dispensed into a 300-cc glass beaker. The mixture is heated to boiling temperature. 12 cc of propylene glycol is added to the mixture. The temperature setting is maintained at 350° C. for 2 minutes.

Step 4. 6 g of hydroxyethyl cellulose is added to the heated mixture of sterile water and propylene glycol. The mixture is stirred continuously while quickly adding the hydroxyethyl cellulose. Once the addition of the hydroxyethyl cellulose is complete; the mixture is stirred for one minute on the heat plate. During this step, the gel will begin to form.

Step 5. The mixture is removed from the heat plate. After removal from the heat plate, the gel will become more viscous. While stirring the mixture, the solution of ethyl alcohol/mometasone is slowly added. The gel will become opaque because the mometasone is insoluble in water. The mixture is returned to the heat plate and stirred for one minute. During this step, the ethyl alcohol will evaporate from the mixture.

Step 6. The mixture is removed from the heat plate. The mixture is continuously stirred while the mixture cools. When the gel cools, the dissolved tobramycin in sterile water solution is slowly added. Stirring continues for 5 minutes.

Step 7. While continuing to stir the medication gel, the entire beaker is placed into a bath of isopropyl alcohol 70% for two minutes to cool the gel.

Step 8. The medication gel is poured into a 60-cc syringe with a luer to luer connector, connecting the opposite end with a 12 cc luer tip syringe.

Step 9. Each 12-cc syringe is filled with 7 cc of gel. The filled syringes are kept in a vertical position. Any air bubbles will collect at the top of the syringe. After 5 minutes of cooling and in a vertical position, 1-2 cc of gel are decanted to remove the air, resulting in a final gel syringe volume of 5-6 cc. Using a sterile cap, the tip of the syringe is secured with the luer sterile cap.

Step 10. The gel filled syringes are stored refrigerated prior to shipping. Gel syringes are shipped under refrigerated conditions, for example with cool ice blocks. Upon arrival the gel is kept refrigerated.

Additionally, 24- and 48-hour inspections are suggested to inspect for suspended particulates. In one embodiment, using a syringe from the prepared lot, approximately 3 cc of gel are decanted at 48 hours to inspect for viscosity. This viscosity observation is further repeated at 72 hours.

### Catheter/Syringe

In one embodiment, the medicated gel may be introduced into the sinuses using a delivery device comprising a catheter, such as a suction catheter, and a syringe connected to one end of the catheter. The syringe may contain a particular amount of the viscous composition (e.g., a hydrogel) and one or more medications and/or other compounds. The catheter may be advanced through the ostium to a paranasal sinus cavity, deploying the viscous composition to the paranasal sinus cavity such that the viscous composition adheres to a cell wall of the paranasal sinus cavity. In some embodiments, viscous composition may be deposited via the catheter by effectuating a plunger on the syringe. The volume of the gel deposited into a given area may depend on the extent of the area affecting the paranasal sinus or the severity of the infection. In some embodiments, as little as 1 cc per affected area has been effective in treatment. In some embodiments, the catheter may include one or more shapes, designs, and/or dimensions.

### Malleable Suction

In other embodiments, a method of delivering a viscous composition comprising one or more medications and/or other components may comprise loading the viscous composition into a delivery device comprising a malleable suction device, wherein the suction device comprises one or more curved sections for advancing the suction device through the ostium to a paranasal sinus cavity and deploying the viscous composition to the paranasal sinus cavity such that the viscous composition adheres to a cell wall of the paranasal sinus cavity. For example, the malleable suction device may include part number MKSK5 Malleable Suction Tube in the Kennedy frontal sinus surgery instrument set manufactured by Integra MicroFrance®.

### Cannula/Syringe

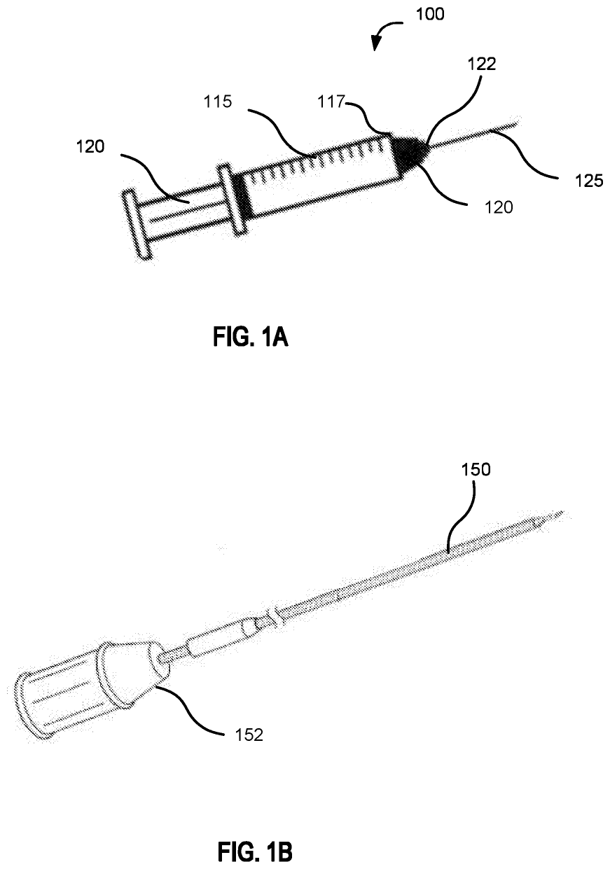

In some embodiments, a method of delivering a viscous composition comprising one or more medications and/or other components may comprise loading the viscous composition into a delivery device. For example, as illustrated in FIG. 1A, the delivery device may include a syringe 100. Syringe 100 may include a barrel 115 comprising a cylindrical tube. The cylindrical tube 115 may be filled with a liquid substance (e.g., a viscous composition) which is discharged through an open end 117, as a plunger 120 is pushed along the inside of barrel 115. and an adapter 125. Open end 117 may be fitted with an adapter 120. Adapter 120 may be configured receive a device configured to direct the flow out of barrel 115. For example, a needle 125 may be received by adapter 120. Needle 125 may be received by adapter 120 via a luer-lock connection 122.

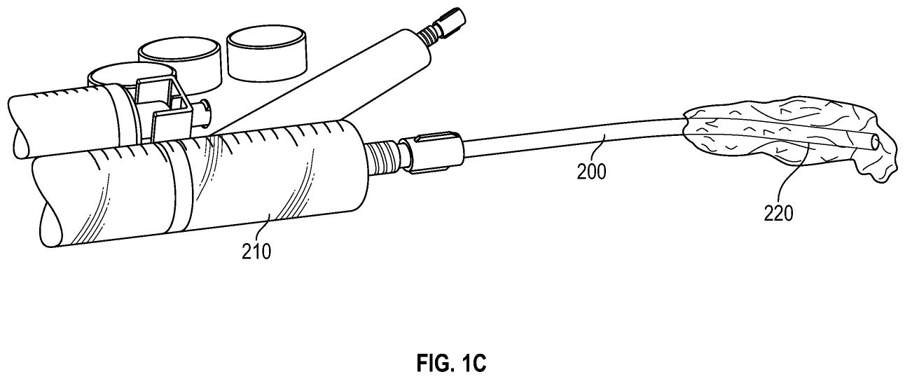

Alternatively, adapter 120 may receive a cannula. For example, as illustrated in FIG. 1B, comprising a cannula 150. Cannula 150 may be fitted inside an adapter 120 (illustrated in FIG. 1A) via a luer-lock connection 152. In some embodiments, cannula 150 may comprise one or more curved sections, for example, for advancing cannula 150 through the ostium to a paranasal sinus cavity and deploying the viscous composition to the paranasal sinus cavity such that the viscous composition adheres to a cell wall of the paranasal sinus cavity. FIG. 2 depicts another example cannula 200 in gel communication with syringe 210 for delivery of the viscous composition 220 to a patient's paranasal sinus cavity.

In some embodiments, the cannula may include one or more curved shapes. In some embodiments, a cannula may be dimensioned based on the intended use and the size of the viscous composition that is being delivered. In some embodiments, an inner surface of the cannula may be defined by an inner diameter. In some embodiments, the diameter of the inner surface may be constant. In yet other embodiments, the inner diameter of the canula may vary throughout the length of the cannula.

In some embodiments, the method of delivering a viscous composition comprising one or more medications and/or other components the delivery device may include a kit, the kit comprising one or more configurations. Referring to FIG. 2, in one configuration, kit 240 may any number of saline syringes, medicated gel syringes for immediate post-operative use, medicated syringes for follow up care visits, and luer-lock cannulas. In some embodiments, each of the components of the kit (i.e., saline syringes, medicated gel syringes, and cannulas) may be sealed in a sterile vacuum-packed clam shell package, which is resealable for refrigerated storage. The number of syringes and cannulas per kit may vary.

Various embodiments have been described with reference to specific exemplary features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the present application, whether or not such embodiments are described and whether or not such features are presented as being a part of a described embodiment. Thus, the breadth and scope of the present application should not be limited by any of the above-described exemplary embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary diagrams and other illustrations. As will become apparent to one of ordinary skill in the art after reading this document, the illustrated embodiments and their various alternatives can be implemented without confinement to the illustrated examples. For example, diagrams and their accompanying description should not be construed as mandating a particular configuration.

1. A medicated gel, comprising:
a sterile viscous gel having a viscosity sufficient to be maintained within a paranasal sinus cavity or nasal passage after insertion; and
a medication dispersed in the sterile viscous gel wherein the sterile viscous gel contains chitosan and wherein the medication comprises mometasone furoate or mometasone furoate monohydrate.

2. The medicated gel of claim 1, wherein the sterile viscous gel is selected from the group consisting of: hydroxyethyl cellulose, hypromellose, methlycellulose, carboxymethlycellulose and other cellulose type polymers.

3. The medicated gel of claim 1, wherein the sterile viscous gel contains time release lipid matrix microspheres.

4. (canceled)

5. (canceled)

6. The medicated gel of claim 1, wherein a viscosity of the gel is between 10 and 300,000 centipoise.

7. The medicated gel of claim 1, wherein the medication is selected from the group consisting of: tobramycin, gentamicin, vancomycin, ceftriaxone, ceftazidime, clindamycin, levofloxacin, ciprofloxacin, mupirocin, azithromycin, clarithromycin, polymyxin B, colistin, sulfamethoxazole/trimethoprim and ertapenem.

8. The medicated gel of claim 1, wherein the medication is selected from the group consisting of: Penicillins; Cephalosporins; Tetracyclines; Macrolides; Quinolones; Fluoroquinolones; Aminoglycosides; Carbapenems; Sulfonamides; Glycopeptides and Polymyxins.

9. The medicated gel of claim 1, wherein the medication is selected from the group consisting of: Fluconazole, Itraconazole, Voriconazole, Posaconazole, Isoconazole, Amphotericin B, Amphotericin B Liposome, and Amphotericin B Lipid Complex.

10. The medicated gel of claim 1, wherein the medication is selected from the group consisting of: betamethasone, fluticasone furoate, fluticasone propionate, ciclesonide, hydrocortisone, budesonide.

11. The medicated gel of claim 1, wherein the sterile viscous gel contains ibuprofen, diclofenac, naproxen, ketoprofen or meloxicam.

12. The medicated gel of claim 1, wherein the sterile viscous gel contains methylglyoxal, Vitamin D3, Vitamin A, silver or gold ions or colloidal suspensions, ethylenediaminetetraacetic acid (EDTA), hyaluronic acid (HA), xylitol, bromelain, quercetin, herbal extracts, or essential oils.

13. A method of delivering the medicated gel of claim 1 into a paranasal sinus cavity or nasal passage, the method comprising:
depositing the medicated gel into the infected paranasal sinus cavity and onto the surrounding tissues.

14. The method of claim 13, wherein depositing the medicated gel comprises applying the medicated gel via a delivery device comprising a syringe and catheter assembly, a syringe and malleable suction assembly, or a syringe and cannula assembly during or after functional endoscopic sinus surgery.

15. The method of claim 13, wherein depositing the medicated gel comprises applying the medicated gel via a balloon catheter during or after balloon sinuplasty.

16. The method of claim 13, wherein depositing the medicated gel comprises applying the medicated gel with pre-soaked pledgets or swabs, and spreading the gel with a swab applicator directly onto the paranasal sinus cavity or nasal passage.

17. The method of claim 13, wherein depositing the medicated gel comprises applying the medicated gel using a pump bottle.

18. The method of claim 13, wherein depositing the medicated gel comprises applying the medicated gel using vials.

19. The method of claim 13, wherein depositing the medicated gel comprises applying the medicated gel using tubes.

20. The method of claim 13, wherein depositing the medicated gel comprises applying the medicated gel using a pressurized can.

21. The method of claim 13, further comprising diluting the medicated gel with saline before depositing the medicated gel, wherein depositing the medicated gel is administered using an irrigating device.

22. A method of delivering the medicated gel of claim 1 into a paranasal sinus cavity or nasal passage, the method comprising:
a. Rinsing a paranasal sinus cavity or nasal passage with sterile saline solution;
b. Removing damaged tissue (debridement) from the paranasal sinus cavity or nasal passage;
c. Rinsing a paranasal sinus cavity or nasal passage with sterile saline solution; and
d. Depositing a medicated gel into the infected paranasal sinus cavity and surrounding tissues or nasal passage.
